(19) United States
(12) Patent Application Publication
(10) Pub. No.: US 2005/0265989 A1
(43) Pub. Date: Dec. 1, 2005

(54) Non-toxic purification and activation of prothrombin and use thereof

(75) Inventors: Even Manseth, Oslo (NO); Svein Olav Fjaera, Sunndalsora (NO); Per Olav Skjervold, As (NO); Ole Rasmus Odegard, Oslo (NO); Frank Roger Brosstad, Oslo (NO); Ragnar Flengsrud, As (NO)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Kald Fisk AS, Bremnes (NO)

(21) Appl. No.: 11/050,442

(22) Filed: Feb. 4, 2005

Related U.S. Application Data
(60) Provisional application No. 60/541,750, filed on Feb. 5, 2004.

(51) Int. Cl. A61K 38/46; C12N 9/74

(52) U.S. Cl. 424/94.65; 435/214

(57) ABSTRACT

The present invention relates to a prothrombin activator for activating prothrombin to thrombin comprising of an extract of fish gills or a combination of fish egg and fish gills. Said activator is suitable for use in medical applications as well as in food products.

### CROSS-REFERENCE TO RELATED APPLICATION

This application claims the benefit of U.S. Provisional Application No. 60/541,750, filed Feb. 5, 2004, the disclosure of which is herein incorporated by reference in its entirety.

### FIELD OF INVENTION

The present invention relates to a method of non-toxic purification of blood thrombins in particular from fish species e.g. salmon. In particular, the present invention pertains to the purification and non-toxic activation of prothrombins to thrombin. The novel method allows for use of the thrombins in medical applications as well as in food products.

### TECHNICAL BACKGROUND

Fibrin sealants, comprising thrombin, fibrinogen and Factor XIII, are effective hemostatic agents, which have found use in surgery and other medical applications. Concerns are however expressed regarding the possibility of disease transmission from human-derived fibrinogen from non-autologous sources, and the bovine thrombin conventionally used. A method for the purification and use of fibrinogen and thrombin from coldwater fish involving toxic substances has been described in U.S. Pat. No. 6,007,811 as an alternative to the conventional fibrin sealants.

Methods known in the art for purifying fibrinogen and thrombin, and for activating the inactive form prothrombin to thrombin, involve chemicals which may be present in the fibrin sealant and which may be unwanted for particular applications.

Further, fibrin sealants derived from fish may be of interest in the food industry for the same reasons as described above. Additionally, the demand for new food products and “convenient food” are increasing and fibrin sealants may find use in such new products. However, presence of chemicals in the fibrin sealant not allowed for human consumption must be avoided.

The inventors of the present invention developed a non-toxic purification method for thrombin, where the activation of prothrombin was performed using an extract obtained from fish. The thrombin obtained by the present invention has been shown to have good clotting effect with high specific activity. The described method of non-toxic activation and purification provides a product, which does not comprise chemicals and toxins and therefore, is useful in the food as well as in the pharmaceutical industry.

### SUMMARY OF THE INVENTION

Accordingly, in the broadest aspect of the present invention a method for producing thrombin is provided, the method comprising the steps of:
(i) drawing whole blood from a donor fish,
(ii) separating plasma from the whole blood,
(iii) extracting prothrombin from the plasma;
(iv) activating the prothrombin to thrombin by subjecting the prothrombin to an extract obtained from a second fish, said extract comprising a prothrombin activator.

In another aspect of the present invention a thrombin preparation obtainable by the above mentioned method is provided.

In yet an aspect of the present invention a prothrombin activator for activating prothrombin to thrombin comprising of an extract of fish gills or a combination of fish egg and fish gills is provided.

In a further aspect of the present invention a method for producing a fibrin sealant as well as the sealant as such obtainable by this method is provided. The method comprising the steps of:
(i) providing a fibrinogen and Factor XIII
(ii) providing a thrombin, where said thrombin has been activated using the prothrombin activator described above, and
(iii) combining the fibrinogen and Factor XIII of step (i) and the thrombin of step (ii) to form the fibrin sealant.

### DETAILED DISCLOSURE

Several types of meat-binding agents are being used in the food industry. A microbial transglutaminase product (Activa™; Ajinomoto, Tokyo, Japan) has been used for some time in the food industry for meat binding. Binding agents based on the coagulation proteins in blood are being used in the meat industry (Fibrimex™; Harimex, Loenen, Netherland).

Such binding agents are also used in human medicine, especially in surgery. (Tisseel™; Immuno AG, Vienna, Austria).

The protein sources for these products are bovine, porcine, and human blood.

Based on recent concerns that a new variant of Creutzfeld-Jacob disease is linked to bovine spongiform encephalopathy (BSE), alternative sources for binding agents are being investigated. In this respect the expanding, intensive fish farming industry constitutes a large new source of muscle protein, but also of by-products such as fish blood. The amount of blood in salmonids is 3-5% of the bodyweight (Hoar and Randall 1969) and the quantity of farmed Atlantic salmon today is close to 1 million metric tons per year. Blood from farmed fish could therefore serve as a substitute for mammalian blood as a source for binding agents, leading to the development of new meat products or for its use in human surgery.

Thrombin is a serine proteinase that is centrally involved in the final step of the coagulation process in blood. Thrombin cleaves the 4 arginyl-glycyl peptide bonds in fibrinogen to produce fibrin monomers, which spontaneously polymerize to form an insoluble fibrin network. Its inactive precursor, prothrombin, belongs to the family of vitamin K-dependent blood-coagulation factors. The single polypeptide chain prothrombin (factor II) is activated to the two-chain alpha-thrombin (factor IIa). This activation is a result of extrinsic or intrinsic activation leading to a complex of factor Xₐ, prothrombin, Ca²⁺, and factor Vₐ on a phospholipid membrane surface. Extrinsic activation of mammalian thrombin is commonly performed using brain, lung, or placental tissue extracts; these are particularly rich sources of tissue thromboplastin. Prothrombin has previously also been activated by the use of enzymatic activities of viper venoms, for example, Echis carinatus (Esnouf 1977).

The vitamin K-dependent proteins have a specific affinity for insoluble barium and magnesium salts. Barium adsorption of prothrombin is therefore, the most widely used initial step to separate these proteins from the other coagulation factors in plasma.

Blood coagulation in fish is fundamentally similar to mammalian blood coagulation and in particular it has been shown that the vitamin K-dependent system in salmon shows similarities with the human coagulation system. A more detailed study of salmon antithrombin III showed that this molecule had high sequence identity and functional similarities with mammalian antithrombins but also important differences that might reflect different organism needs.

Varies studies describing the purification of thrombin from Atlantic salmon has recently proposed said purified thrombin from salmon as an alternative to mammalian thrombins in fibrin sealants. In such a procedure snake venoms were used for activation of prothrombin in addition to other non-consumable ingredients. Thus, industrial application of salmon thrombin requires production and activation processes that are easy to scale up, economically feasible, and compatible with the use of the end product as a food additive or in human medicine.

With the goal of developing a food ingredient or for the use in medicine, such as human or animal surgery, the inventors of the present invention investigated principles of purification of a highly active thrombin using an activator obtained from a donor fish to activate prothrombin to thrombin. In one embodiment of the present invention only nontoxic and consumable components and biomolecules primarily from fish, such as salmon, are used in the process and during the downstream process.

In yet an embodiment of the present invention porcine heparin is used in the chromatography step and the search for alternative heparin sources are in progress. Preliminary experiments also showed that the ion exchanger SP-Sepharose from Amersham Pharmacia Biotech (Uppsala, Sweden) might be a good alternative to the heparin-Sepharose column used in this work. The inventors of the present invention investigated the purification of salmon thrombin together with the study of different parameters affecting the prothrombin adsorption to BaSO₄. Considering this embodiment, in order to provide a potential food ingredient or a product useful in medicine the inventors wanted to avoid activation of prothrombin to thrombin by the use of brain tissue, consequently alternative sources for tissue activation of salmon prothrombin were investigated.

Therefore the inventors studied the activation of prothrombin using Atlantic salmon eggs and gills, including parameter optimalization and development of a protocol that seems feasible for industrial application. Since studies in mammals have shown that thrombin may occur in various forms with varying biological activities the inventors have determined the N-terminal sequences of purified activated thrombin to reveal possible heterogeneities. This determination may be relevant because little is known about thrombin processing in fish and because a new prothrombin activation mixture was used.

The present invention shows in one part a study of the activation, purification and characterization of Atlantic salmon (Salmo salar) thrombin, to be used as a possible source for binding agents in the food industry or as a suitable binding agent in medicine e.g. in human or animal surgery.

Thus, in an embodiment of the present invention a mixture of eggs and gills from salmon provided and optimized conditions for the adsorption, elution and the activation step are presented. The purified thrombin clotted e.g. bovine fibrinogen with a specific activity of 1423 U/mg. Sequence data are presented and compared to other species. The method of non-toxic activation and purification described by the present inventors will allow donor fish thrombin to be used in the food as well as in the pharmaceutical industry.

In a preferred embodiment of the present invention a method for producing thrombin is provided. The method comprising the steps of: (i) drawing whole blood from a donor fish, (ii) separating plasma from the whole blood, (iii) extracting prothrombin from the plasma; and (iv) activating the prothrombin to thrombin by subjecting the prothrombin to an extract obtained from a second fish, said extract comprising a prothrombin activator.

Prothrombin obtained according to the present invention or provided from any kind of fish or other species than fish may be subjected to a prothrombin activator obtained from fish. In the present context the term “prothrombin activator” is used to indicate an extract obtained from a second fish, identical to, same as or different from the donor fish, which is capable of assisting the conversion of prothrombin to thrombin. In the present context the term “assisting” relates to the need for the prothrombin activator to be present in order to have prothrombin converted to thrombin. The features mentioned in respect of method of producing thrombin apply mutatis mutandis to the prothrombin activator.

In the present context the term “second fish” relates to a fish from which the prothrombin activator may be been obtained. In an embodiment of the present invention the fish is selected from the group consisting of a salt-water fish, a brackish-water fish and a fresh-water fish. The group of salt water fish may include fish belonging to the genus Salmonidae, including Atlantic salmon (Salmo salar), but also rainbow trout (O. mykiss). One especially preferred group are farmed fish of any genus, but especially from the genus Salmonidae. These species are selected because they are reared in large numbers, and individuals grow large enough (over one kilogram) so that blood can be drawn easily. Other farmed cold-water fishes, such as halibut or cod, may be used as donor fish and might satisfy all the above criteria. In an embodiment of the present invention the fish is a domesticated, farmed fish and or a cold water fish.

In an embodiment of the present invention the activation process in step (iv) may be carried out at 0-40° C., preferably at 2-30° C., such as at 3-25° C., e.g. at 4-20° C., or or at 13-17° C. Furthermore, the activation may be carried out under gentle stirring for a period of 30 second to 48 hours, such as 10 minutes to 5 hours, e.g. 25 minutes to 3 hours, such as 45 minutes to 2 hours, e.g. 25 minutes to 1 hours, such as 1 hour to 5 hours, e.g. 1 hour to 2 hours. In an embodiment of the present invention the activation may be carried out under gentle stirring for a period of about 30 minutes, such as about 45 minutes, e.g. about 1 hour, such as about 2 hours. After completion of the activation process as described above the activation process may be terminated. Preferably, the termination of the activation process may be performed by addition of sodium oxalate in a concentration of 0.01 to 0.5 M, preferably 0.04-0.1 M.

In an embodiment of the present invention the activated thrombin may be isolated by centrifugation and/or filtration. If centrifugation is performed the supernatant obtained is filtered to isolate the activated thrombin.

In the present context the term “donor fish” relates to a fish from which thrombin and/or prothrombin may be been obtained. The way of obtaining the thrombin and/or prothrombin may be by any known suitable method of isolating components from blood. In an embodiment of the present invention the fish is selected from the group consisting of a salt-water fish, a brackish-water fish and a fresh-water fish and the group of salt water fish may include fish belonging to the genus Salmonidae, including Atlantic salmon (Salmo salar), but also rainbow trout (O. mykiss). One especially preferred group are farmed fish of any genus, but especially from the genus Salmonidae. These species are selected because they are reared in large numbers, and individuals grow large enough (over one kilogram) so that blood can be drawn easily. Other farmed cold-water fishes, such as halibut or cod, may be used as donor fish and might satisfy all the above criteria. In an embodiment of the present invention the fish is a domesticated, farmed fish and or a cold water fish.

In a preferred embodiment of the present invention the new thrombin preparation provided by the new method according to the present invention may be combined with fibrinogen and Factor XIII to form a fibrin sealant. Such fibrinogen may be obtained from a fish or a mammal from blood plasma through a precipitation reaction with ammonium sulphate or ethanol. In an embodiment of the present invention the mammal is selected from the group consisting of a human being, a cow, a pig, a sheep, a goat and a horse. Factor XIII, which is a transglutaminase is obtainable from blood plasma in the same fraction as fibrinogen through for instance an ethanol precipitation reaction. Alternatively, both fibrinogen and Factor XIII are commercially available from Sigma.

In an embodiment of the present invention the fibrinogen is obtained from the plasma which has been separated from whole blood obtained from a fish or a mammal. In the case the fibrinogen is obtained from a fish then this fish may be of the same species or of different species as the donor fish.

In an embodiment of the present invention the combination of thrombin and fibrinogen may further be supplemented by adding a salt, preferably a calcium salt to form the fibrin sealant.

The isolation of the prothrombin and/or fibrinogen from the fish may be initiated by starving the donor fish prior to drawing whole blood from the donor fish. In an embodiment of the present invention the donor fish is starved for about 36 hours, such as 24 hours, e.g. 20 hours, such as 16 hours and e.g. 10 hours. In another embodiment of the present invention the donor fish is starved for a period in the range of 0-48 hours, such as in the range of 5-40 hours, e.g. in the range of 10-36 hours, such as in the range of 10-30 hours, e.g. in the range of 10-24 hours, such as in the range of 15-24 hours, e.g. in the range of 16-20 hours. The fish may be anesthetized to a loss of reflex activity by use of any suitable means such as e.g. by bubbling carbon dioxide through the water.

Subsequently, the whole blood may be drawn from the donor fish. Any known method for obtaining whole blood from e.g. a fish may be used. In an embodiment of the present invention whole blood may be drawn from the caudal vein or artery of the fish by known methods such as using a needle and syringe, vacuum tube, or other vacuum device. In another embodiment, the fish is gutted and the blood is collected by any suitable means. With all of these devices, one part of 0.2 M sodium citrate or 0.1 M solution of sodium oxalate is added to nine parts of whole blood as an anticoagulant. The whole blood is held at about 1° C. to 4° C. for no more than about four hours before centrifugation.

After the whole blood has been obtained plasma may be separated from the whole blood. This separation of plasma may be performed at about 25° C., such as at about 15° C., e.g. at about 10° C., such as at about 5° C., e.g. at about 4° C., such as at about 3° C., e.g. at about 0° C. In an embodiment of the present invention the separation of plasma may be performed at a temperature in the range of 0-25° C., such as in the range of 1-20° C., e.g. in the range of 2-15. such as in the range of 4-10° C., e.g. in the range of 5-10. The separation further involves filtration, chromatographic separation and/or centrifugation at least about 1000 g for about 10 minutes.

The separation of blood cells and plasma may preferably be done at about 4° C., and at least about 1000 g for about ten minutes. The plasma may then be frozen, preferably at −20° C., or extracted immediately.

In a preferred embodiment of the present invention the prothrombin activator comprises an extract of fish gills or a combination of fish egg and fish gills. In yet an embodiment of the present invention the extract comprises the prothrombin activator in a ratio between fish egg and fish gills in the range of 0:1 to 25:1, such as in the range of 0:1 to 15:1, e.g. in the range of 0:1 to 9:1, e.g. in the range of 1:1 to 25:1, such as in the range of 1:1 to 15:1, e.g. in the range of 1:10 to 10:1, such as in the range of 1:5 to 5:1, e.g. in the range of 1:3 to 3:1. In yet another embodiment the ratio between fish egg and fish gills is in the range of 0:1 to 1:50, such as in the range of 0:1 to 1:25, such as in the range of 0:1 to 1:15, e.g. in the range of 0:1 to 1:1, e.g. in the range of 1:1 to 1:25, In a further preferred embodiment of the present invention the extract comprises the prothrombin activator in a ratio between fish egg and fish gills of 1:5, such as 1:4, e.g 1:3, such as 1:2, e.g 1:1, such as 2:1, e.g 3:1, such as 4:1, e.g 5:1.

Extraction procedures are preferably known methods currently used for bovine thrombin and fibrinogen.

In an embodiment of the present invention the extraction of prothrombin may preferably be performed by first using BaSO₄ or Ca₃ (PO)₄. Activation of the prothrombin to thrombin and subsequent extraction methods are preferably carried out by subjecting prothrombin to the prothrombin activator as described herein. Fibrinogen may be extracted from the supernatant using the ammonium sulfate methods described by Silver et al. (1995). One part of a saturated (4.5 M) solution of ammonium sulfate at about 4° C., is added to three parts of the supernatant. Fibrinogen may also be extracted by using the ethanol precipitation method described by Murtaugh et al. (1973). Then the precipitated protein fraction is centrifuged, preferably at about 14,000 g at about 4° C. for about 8 minutes. The fibrinogen is resuspended in a citrate buffered saline (pH 7.4) at room temperature at a concentration of 2.5 mg/ml. The thrombin is resolublized in 40 mM calcium chloride at a concentration of 0.25-1 NIH units/ml. Commerically available bovine or human thrombin may be used at similar concentrations with the fish fibrinogen to achieve similar results, but without the degree of safety provided by the fish thrombin.

The new thrombin preparation according to the present invention and obtained from the above method may preferably be essentially free from artificial chemicals—the term “artificial chemical” relates to chemicals not originally present in the fish extract—and free from toxins conventionally added e.g. for activating prothrombin to thrombin. Thus, the features mentioned in respect of the method of producing thrombin above apply mutatis mutandis to the thrombin preparation according to the present invention.

A “thrombin preparation” may be any preparation of thrombin in solid or fluid form, such as for instance a mixture, e.q a dry mixture, or a solution, a suspension or an emulsion.

In a preferred embodiment of the present invention the thrombin preparation according to the present invention may be combined with the fibrinogen to produce a food product in particular a fish product.

In yet a preferred embodiment of the present invention the thrombin preparation according to the present invention may be combined with the fibrinogen on a mammalian tissue, preferably a human tissue to work as an effective hemostatic agent with numerous applications in cardiac, thoracic, plastic, and neuro surgery; skin grafting, repair of bony defects, and treatment of gastric ulcers. Further applications may be the need for a biodegradable tissue sealant that serves to diminish bleeding or serosol leakage, or to provide additional strength to surgical anastamoses.

In one such embodiment the two components may be applied to the wound or leakage simultaneously using a commercially available double syringe or spray applicator.

In an alternative embodiment of the present invention a method for producing a fibrin sealant may be provided. The method comprising the steps of: (i) providing a fibrinogen, (ii) providing a thrombin, where said thrombin has been activated using a prothrombin activator for activating prothrombin to thrombin comprising of an extract of fish gills or a combination of fish egg and fish gills, and (iii) combining the fibrinogen of step (i) and the thrombin of step (ii) to form the fibrin sealant. The features mentioned in respect of the method of producing thrombin apply mutatis mutandis to this method of producing a fibrin sealant.

In an embodiment of the present invention at least one of the fibrinogen or the thrombin is extracted from plasma and said plasma is separated from whole blood which is drawn from a donor fish or a mammal.

The new sealant provided may comprise thrombin, fibrinogen and a prothrombin activator for activating prothrombin to thrombin comprising of an extract of fish gills or a combination of fish egg and fish gills. The features mentioned in respect of the method of producing thrombin above apply mutatis mutandis to the sealant.

The advantages of the fish-derived substances include the following:

A. Safety

The safety advantages of deriving the components of a fibrin sealant, fibrinogen and thrombin, from fish blood can be best understood in the context of the evolutionary biology of these fish. The fishes as a group (phylum) are widely separated from mammals, and as such, their disease organisms have evolved on separate paths. These differences are exemplified in standard laboratory methods in which various fish cell lines must be used to propagate fish viruses, as mammalian cell lines are used for mammalian viruses. Another difference is temperature. In coldwater fish such as salmon or trout, their maximum body temperature is the same as the water in which they live normally between about 0° C. and 18° C., a temperature range nearly 30° C. below that of humans or most other mammals. Therefore, these fish have few, if any, infectious agents that can survive in humans. These are just some of the manifestations of the wide evolutionary distance between fish and mammals that result in safety from infectious agents.

B. Control of Source

Clotting factors derived from human or bovine blood may be inconsistent in quality due to variations in both genetics and environment of the donors. In contrast, domesticated, farmed fish that serve as blood donors are well-defined as to diet, habitat, reproductive status, life history, and genetic background. The degree of control that aquaculture provides for these donor animals results in improved uniformity of product. Unlike autologous cryoprecipitate, pre-tested salmonid fibrinogen offers consistent concentrations and generally greater quality control.

C. Rapid Clotting Time

Clotting time (thrombin time) in salmon and trout plasma was measured by standard coagulation laboratory techniques using bovine thrombin. Compared to a Human Reference Range of 12-16 seconds, mean salmon thrombin time was 6.8 seconds and trout thrombin time was 7.1 seconds.

For applications requiring a fibrin sealant, the present invention, derived from fish, can be used with similar efficacy, and advantages in safety, quality control, and product content over the human/bovine-derived fibrin sealants currently in use.

Further Embodiments of the Present Invention.

In an embodiment of the invention relates to a non-toxic method for the purification of prothrombin from fish. Preferably, the method involves the use of BaSO₄ adsorption and heparin-Sepharose affinity chromatography or SP-sepharose chromatography.

It is appreciated, that the prothrombin may be purified from any suitable fish derived from the group consisting of a salt-water fish, a brackish-water fish and a fresh-water fish. The group of salt water fish may include fish belonging to the genus Salmonicidae, including Salmo salar or white fish such as cod or halibut.

Further it should be understood, that the expression “prothrombin” covers all possible forms of prothrombin which, upon activation, result in a preparation with a potential to clot fibrinogen.

In useful embodiments the method involves a non-toxic washing procedure, after BaSO₄ adsorption, with NaCl at room temperature, and an elution of prothrombin from BaSO₄ using a sodium-citrate buffer. Preferably, NaCl is used at a concentration about 0.15 M. A preferred concentration of the sodium-citrate buffer is 0.16 M.

The described method of non-toxic activation and purification will allow salmon thrombin to be used in the food as well as in the pharmaceutical industry.

In yet an embodiment, the invention provides a method for activation of prothrombin using a mixture of eggs and gills from salmon. The eggs and gills may be obtained from any teleost fish species, preferably from trout, cod, halibut, carp, salmon. In a presently preferred embodiment, the eggs and gills are obtained from salmon or cod.

The mixture may be provided such as to obtain a ratio of eggs and gills in the range of from 0:9 to 9:0, preferably the egg/gills ratio is 1:3. The optimal activation temperature is about 15° C. and the activation is preferably stopped using a non-toxic component such as e.g. sodium oxalate.

In still a further embodiment, the invention provides a purified thrombin preparation. The preparation clotted bovine fibrinogen with a specific activity in the range of 50-100,000 U/mg). However, even small amounts of thrombin in a fibrinogen preparation will result in clotting. Thus, amounts in the range of 0.01-0.1 U/mg will result in clotting of fibrinogen.

It is contemplated that the thrombin preparation according to the present invention will be suitable for medical use as well as for use in the food, feed and pharmaceutical industry.

The invention will be further described in the following non-limiting figures and example.

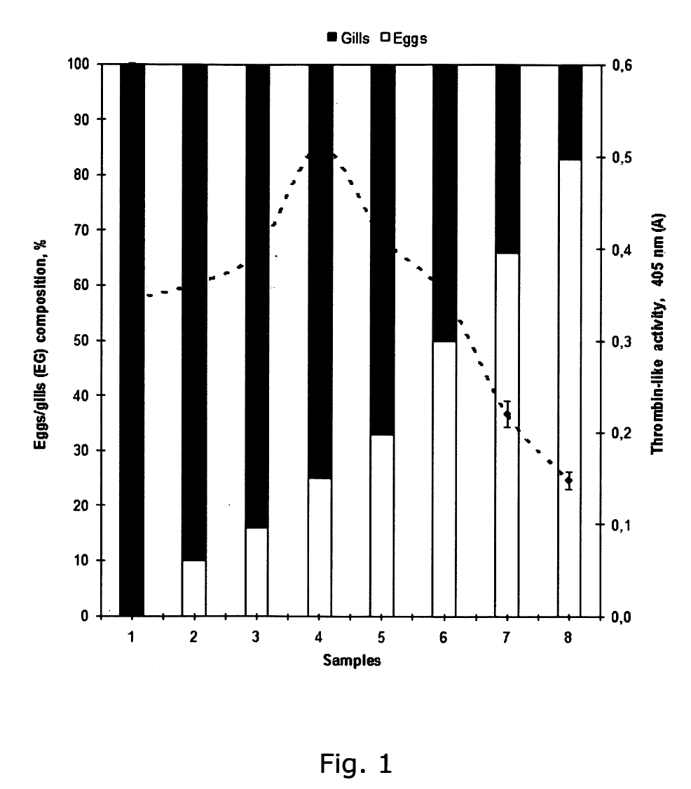

FIG. 1: Effect of EG-composition on thrombin activation. Prothrombin samples during activation consisted of 0.018 M sodium citrate, 0.15 M NaCl, 0.025 M CaCl₂, 2% eggs-gills mixture with varying EG-composition. Samples were activated for 15 min at 15° C. The line indicates the relation between thrombin-like activity and EG-composition. Bars indicate standard error of the mean. Measurements are based on the mean absorption value of 3 measurements for each sample with identical composition at each EG-composition.

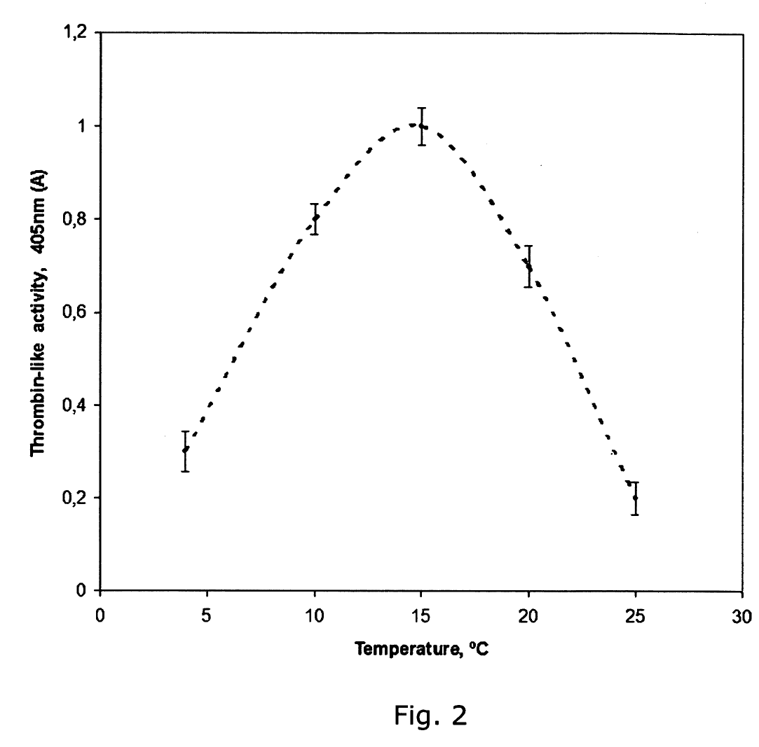

FIG. 2: Effects of temperature on activation of salmon thrombin. Prothrombin samples during activation consisted of 0.018 M sodium citrate, 0.15 M NaCl, 0.025 M CaCl₂, 2% eggs/gills mixture (1:3, v:v). Samples were activated at different temperatures for 15 min. The line indicates the relation between thrombin-like activity and temperature. Bars indicate standard error of the means. Measurements are based on the mean absorption value of 3 measurements for each sample with identical composition at each temperature.

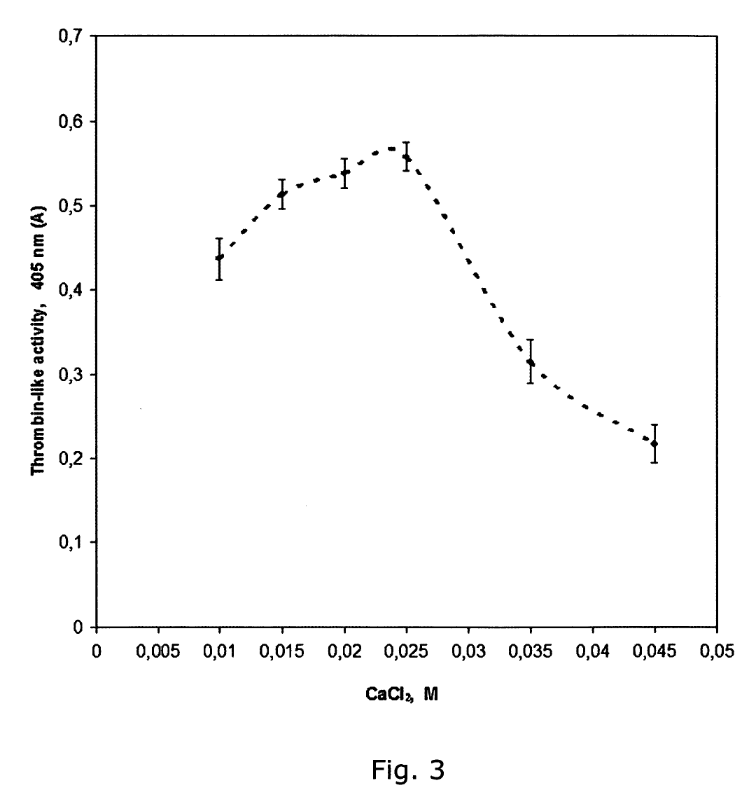

FIG. 3: Shown is the influence of varying CaCl₂ concentration on activation of salmon thrombin. Prothrombin samples during activation consisted of 0.018 M sodium citrate, 0.15 M NaCl, 2% eggs/gills mixture (1:3, v:v). Samples were activated with different CaCl₂ concentration for 15 min at 15° C. The line indicates the relation between thrombin-like activity and [CaCl₂]. Bars indicate standard error of the mean. Measurements are based on the mean absorption value of 3 measurements for each sample with identical composition at each CaCl₂ concentration.

### EXAMPLES

### Example 1

### MATERIALS AND METHODS

Reagents and Chemicals

Bovine thrombin (T4648) and fibrinogen (F8630) were purchased from Sigma Chemical Co. (St. Louis, Mo., U.S.A.). SDS-PAGE Broad Range Unstained Precision Standard was purchased from BioRad (Hercules, Calif., U.S.A.). Substrate H-D-Phe-Pip-Arg-pNA.2HCl (S-2238) was obtained from Chromogenix (Lexington, Mass., U.S.A.). The Micro BCA Protein Assay Reagent including bovine serum albumin as the standard was purchased from Pierce (Rockford, Ill., U.S.A.).

Heparin Sepharose 6 Fast Flow from porcine origin was purchased from Amersham Pharmacia Biotech (Uppsala, Sweden). All other reagents were the best analytical grades available. All aqueous solutions were made from Milli-Q-purified water.

Blood Collection and Plasma Preparation

Atlantic salmon from Aqua Gen breeding program were reared in sea cages for 2.5 y (3-6 kg) at Bremnes Seashore, Norway. Blood was drawn from the caudal vein after anaesthetization by immersion in water cooled to 2° C. by dry ice and with sodium oxalate added to give a final concentration of 0.01 M. The blood was kept on melting ice for less then 1 h before centrifugation (1,600×g, 6 min, 4° C.) by using a MSE GF-8 centrifuge with a universal swing-out head (Sussex, England). The separated plasma was immediately pooled in 500-mL aliquots and stored overnight on melting ice.

Preparation of the Mixture of Salmon Eggs and Gills

Fresh eggs and gill filaments usually from 4 Atlantic salmons (3-6 kg) were washed separately in Milli-Q-water at 4° C. Eggs and gills (EG, wt/wt) were mixed at different ratios; 0:1, 1:9, 1:5, 1:3, 1:2, 1:1, 2:1 and 5:1. One part of egg/gill-mixture was added to one part of Milli-Q-water and homogenized for 5 min on ice with an Ultra-Turrax® at 2000 rpm avoiding temperatures above 10° C. The mixtures were kept as 5-mL aliquots at −80° C.

Determination of Thrombin-Like Activity

The basic enzymology of coagulation and the kinetics of protease action on chromogenic substrates were adhered to for the determination of thrombin activity using the chromogenic substrate S-2238 (Christensen 1980; Rob and others 1997). Thrombin-like activity was monitored as A_{405 nm}. The eluted fraction from the heparin-Sepharose column was assayed for thrombin-like activity using S-2238 as substrate; 500 μL containing 0.04 M Tris-HCl buffer (pH 8.3), 0.1 M NaCl and 0.2 mM S-2238 was maintained at 25° C. for 3 min, then 25 μL thrombin sample was added; ΔA₄₀₅ was measured after 30 s (3 min; when testing thrombin activation parameters). Thrombin activity was calculated by using bovine thrombin as a reference (10-150 U/mL).

Clotting Activity

The clotting activity of the resulting salmon thrombin preparation was measured using bovine fibrinogen as a substrate. Fibrinogen (20 mg) was dissolved in 4 mL of 0.15 M NaCl. A portion (0.2 mL) of this solution was added to an uncoated glass clotting tube and was maintained at 25° C. for 3 min. The thrombin preparation was diluted 1:10 in a 0.06 M Tris-HCl buffer (pH 7.5) plus 0.09 M NaCl, and then 0.1 mL of the diluted sample was added to the uncoated glass clotting tube to mix with fibrinogen. The coagulation was recorded by visual inspection.

Determination of Protein Content and Thrombin Recovery

The protein concentrations were determined by using the (microtiter plate) Bicinchinoic Acid (BCA) assay kit, with bovine serum albumin as the standard protein. To compute thrombin recovery (%), total thrombin activity of the activated BaSO₄ eluate and the recovered eluate from heparin-Sepharose chromatography was divided by the total thrombin activity of the corresponding activated starting plasma.

Determination of Free Ca²⁺ Concentration

Calcium was determined using an ion-selective electrode (E1001) and a reference electrode (E733CA) from Radiometer (Copenhagen, Denmark).

Polyacrylamide Gel Electrophoresis and Blotting

Vertical slab gels (1 mm thick, 12.5% acrylamide separating gel, and a 2.7% acrylamide stacking gel) were used as described for the second dimension electrophoresis in the two-dimensional electrophoresis system of O'Farrell (1975) with the modifications described by Flengsrud and Kobro (1989). The modification of the silver staining (Blum and others 1987) was used. Blotting was performed as described by Flengsrud (1993).

Protein Sequence Analysis

For amino acid sequencing PVDF-electroblotted bands were cut out and placed into a 477 A protein sequencer connected to a 120 A PTH amino acid analyzer from Applied Biosystems (Foster City, Calif., U.S.A.) at The National Centre for Microsequencing and Synthesis of Polypeptides, Biotechnology Centre of Oslo, University of Oslo. Protein sequence results were compared with the public domain sequence databases using the analysis tool described by Pearson and Lipman (1988) and compared with the other characteristics of the highest scoring proteins.

Barium Sulfate Adsorption of Salmon Prothrombin

Routinely, 1000 mL of fresh 0.01 M sodium oxalate plasma was mixed with 100 g BaSO₄ under slow stirring. The BaSO₄ adsorption of prothrombin was examined within 10-60 min using temperatures 1-20° C. The insoluble BaSO₄ was sedimented by centrifugation (12000+g, 15 min) at corresponding temperatures using a Beckman (Fullerton, Calif., U.S.A.) Coulter Avanti J-25 (JA-14 fixed angle rotor) centrifuge. The prothrombin-containing pellet was added to 1000 mL of either 0.15 M NaCl or water. Each suspension was stirred for 5 min at room temperature and 4° C. using an Ultra-Turrax® at 2000 rpm. After centrifugation as above, the supernatant was discarded and the washing procedure repeated 3 times. The adsorbed proteins were eluted from the BaSO₄ pellet using 30 mL of sodium citrate with concentrations from 0.03 to 0.2 M unbuffered citrate (pH 8.3) or citrate buffered (pH 7.3) with citric acid. The suspension was stirred 15 min using an Ultra-Turrax® at 500 rpm. The supernatant containing prothrombin was separated from BaSO₄ by centrifugation as described above. It was then diluted with a solution of NaCl to a final concentration of 0.025 M sodium citrate, 0.15 M NaCl, and adjusted to pH 7.3 with citric acid.

Prothrombin Activation.

The prothrombin solution was activated using varying mixtures of eggs and gills and to a final concentration of 2% (w/v). The concentration of CaCl₂ varied from 0.01 to 0.045 M. Activation was carried out in a temperature range of 4-25° C. under gentle stirring for 1 h. Subsequently, 0.06 M sodium oxalate was added using gentle stirring for 2 min. The solution was centrifuged (12,000×g, 10 min, 4° C.) and the supernatant filtered through a 0.22 μm Millex GP-50 filter from Millipore (Molsheim, France).

Heparin Sepharose Affinity Chromatography

The activated prothrombin (routinely 105 mL) was applied to a column (2.6×15 cm) of heparin-Sepharose 6 Fast Flow, previously equilibrated using buffer A containing 0.025 M citrate buffer plus 0.15 M NaCl, pH 7.3, at 4° C. The column was first washed exhaustively with the equilibration buffer and then with 0.025 M citrate buffer plus 0.45 M NaCl, pH 7.3. Elution was performed with a 0.025 M citrate buffer plus 2.5 M NaCl, pH 7.3. The eluted material was analyzed by SDS/PAGE and assayed for thrombin activity.

### RESULTS AND DISCUSSION

Preliminary Experiments for BaSO₄ Adsorption of Prothrombin

Prothrombin from salmon plasma was prepared using a modification of the method described by Biggs (1976). BaSO₄ adsorption for 30 min at 4° C. gave the highest prothrombin yield. We compared distilled water (Biggs 1976) with 0.15 M NaCl as washing reagents. The distilled water was shown to contain some prothrombin after washing. The washing procedure with 0.15 M NaCl at room temperature gave no loss of the prothrombin. The elution of prothrombin from BaSO₄ gave the highest yield at 0.16 M sodium citrate in an unbuffered system (pH 8.3).

Activation of Prothrombin with Eggs/Gills Mixture

Tissue activation of thrombin will depend on different parameters, such as pH, Ca²⁺, ion strength, and content of tissue factors and lipids. Gills showed tissue factor-like (thromboplastin) activity, while eggs did not (data not shown). Preliminary experiments showed that mixtures of eggs and gills had no amidolytic activity on S-2238. FIG. 1 shows that using only gills as tissue activator gave an A_{405 nm} of 0.34. Increasing amounts of salmon eggs in the gills mixture resulted in increased thrombin-like activity up to A₄₀₅ₙₘ of 0.51. This is being proposed as the optimum composition of this eggs-gills mixture (1:3, w/w) as a tissue activator. Further increase of eggs reduced the final thrombin activity. In this activation mixture of eggs and gills, the ratio of tissue factor from gills and components from eggs affect the activation of thrombin.

Activation at Different Temperatures

Temperature affects both activity and stability of enzymes, meaning that the temperature-activity profile of an enzyme reflects activity and stability effects. The Atlantic salmon usually lives in an environment of 4-20° C. and the growth optimum for reared salmon is 14-16° C. (Austreng and others 1986). At 4° C., salmon thrombin had an A_{405 nm} of 0.3 (FIG. 2). With an increase in temperature, the thrombin activity increased from 4° C. to 15° C. The highest thrombin activity at 15° C. had an A_{405 nm} of 1.0. The activity gradually decreased when the activation temperature became higher than 15° C. Under the conditions given here the optimum is at 15° C., while human and bovine systems show an optimum activation temperature around 37° C. Atlantic salmon is a cold-adapted teleost fish so the interaction between the enzymes involved during the activation of prothrombin is probably at its optimum at 15° C., within the temperature optimum for growth. However, the stability optimum for salmon thrombin alone is 26° C. (Michaud and others 2002). This shows a different temperature optimum for the activation of prothrombin and for the thrombin stability.

Effect of Varying CaCl₂ Concentration on Activation

Activation of thrombin is dependent on the free Ca²⁺ level in the sample. The ratio between the chelator citrate and Ca²⁺ will directly affect the free Ca²⁺ level in the activation mixture. Salmon thrombin activity at 0.01 M CaCl₂ had an A_{405 nm} of 0.44 (FIG. 3). The highest thrombin activity had an A_{405 nm} of 0.56 at 0.025 M CaCl₂ while a further increase of CaCl₂ reduced the final thrombin activity. The free Ca²⁺ concentration at the highest thrombin activity was measured as 8.5 mM at pH 5.7 (data not shown).

The amount of prothrombin in human and bovine plasma, which can be activated, corresponds to an activity of approximately 200 U/mL. Results in this work showed 240 U/mL of thrombin in activated salmon plasma (Table 1). The total protein content in salmon plasma has been reported to be 41.6-56.6 mg/mL (Sandnes and others 1988); it was shown to be 38 mg/mL in the salmon plasma used in this work. This thrombin preparation clotted bovine fibrinogen showing a specific activity of 1423 U/mg.

TABLE 1
Purification of salmon alpha-thrombin by BaSO₄ adsorption and heparin-
Sepharose chromatography

| | Total | Total | Specific | Purifi- | |
|---|---|---|---|---|---|
| Purification | activity | protein | activity | cation | Yield |
| step | (U) | (mg) | (U/mg) | factor | (%) |
| Activated plasma | 240 ± | 38 ± | 6 ± 0.2 | 1 | 100 |
| | 8 × 10³ | 9 × 10³ | | | |
| Activated BaSO₄ | 524 ± | 80 ± | 66 ± 1.4 | 11 | 22 |
| eluate | 9 × 10² | 2 × 10¹ | | | |
| Heparin-Sepharose | 427 ± | 29 ± 1 | 1423 ± 22 | 237 | 18 |
| | 7 × 10² | | | | |

Sequence of Alpha-Thrombin from Atlantic Salmon

The results from the protein sequencing are given in Table 2. The molecular weight (MW) and the sequences of T2 and T3 were consistent with 2 versions of the thrombin heavy chain, one of them apparently being truncated. The MW of T1 suggests that this band contains a mixture of light and heavy chains. The sequencing yielded two amino acids in each step (see legend to Table 2) that indicated that, indeed, a light and heavy chain was being sequenced simultaneously. Apparently, the reductive conditions used for sample preparation were not sufficiently stringent to break the disulfide bonds connecting the two chains. Note that the heavy chain in T1 has a three-residue extension at its N-terminus, compared to the heavy chain in T2 and T3. The T1 band yielded only T in the 6th sequencing round. This could mean that the heavy chain in T1 has another residue at this position (T) than the heavy chain in T2 and T3 (which have a D). However, amino acid concentrations were low and it is thus possible that the expected D simply was not detectable. Taken together, the sequencing results suggest that this thrombin is an alpha-thrombin with heavy and light chains homologous to the thrombin described from other species, and with an apparent similar activation from prothrombin. The sequence results and MW of T4 and T5 indicated non-thrombin polypeptides, most likely histones from the eggs in the activation mixture. A further improved purification of salmon thrombin is in progress; it is expected to give significantly purer thrombin and more detailed characterization. However, we believe that the protocol presented here results in sufficient activity and purity for the use of this thrombin product in the food industry.

### CONCLUSIONS

Prothrombin was recovered from plasma by BaSO₄ adsorption and activated using the found optimal conditions before separation with heparin-Sepharose affinity chromatography. This proved to be an efficient method to isolate thrombin from salmon plasma with a satisfactory specific activity of 1423 U/mg protein. The thrombin preparation was done using nontoxic and consumable components and biomolecules exclusively from salmon. Some sequence data on light and heavy chain alpha-thrombin from salmon are presented.

[image]

### REFERENCES

U.S. Pat. No. 6,007,811
Sandnes and others 1988
Murtaugh and others 1973
Michaud and others 2002
Austreng and others 1986
Biggs 1976
Pearson and Lipman (1988)
Flengsrud (1993).
Flengsrud and Kobro (1989).
Blum and others 1987
O'Farrell (1975)
Christensen 1980
Rob and others 1997
Esnouf 1977
Hoar and Randall 1969
Silver et al. (1995)

1. A method for producing thrombin, the method comprising the steps of:
(i) drawing whole blood from a donor fish,
(ii) separating plasma from the whole blood,
(iii) extracting prothrombin from the plasma;
(iv) activating the prothrombin to thrombin by subjecting the prothrombin to an extract obtained from a second fish, said extract comprising a prothrombin activator.

2. The method according to claim 1, wherein the thrombin is combined with fibrinogen and Factor XIII to form a fibrin sealant.

3. The method according to claim 2, wherein the fibrinogen and/or Factor XIII is obtained from a fish or a mammal.

4. The method according to claim 3, wherein the mammal is selected from the group consisting of a human being, a cow, a pig, a sheep, a goat and a horse.

5. The method according to claim 2, wherein the fibrinogen is obtained from the plasma.

6. The method according to claim 1, wherein the donor fish and the second fish are of the same species.

7. The method according to claim 1, wherein the donor fish are domesticated, farmed fish.

8. The method according to claim 1, further including starving the donor fish for about twenty-four hours prior to drawing whole blood from the donor fish.

9. The method according to claim 1, wherein separating plasma from the whole blood is performed at about 4° C. and includes centrifugation at least about 1000 g for about ten minutes.

10. The method according to claim 1 further including adding calcium to the thrombin and the fibrinogen to form the fibrin sealant.

11. The method according to claim 1, wherein the donor fish is a cold water fish.

12. The method according to claim 11, wherein the cold water fish is selected from the group consisting of a salt-water fish, a brackish-water fish and a fresh-water fish.

13. The method according to claim 1, wherein combining the thrombin and the fibrinogen and Factor XIII includes applying the thrombin and the fibrinogen and Factor XIII to a food product.

14. The method according to claim 13, wherein the food product is a fish product.

15. The method according to claim 1, wherein combining the thrombin and the fibrinogen Factor XIII includes applying the thrombin and the fibrinogen and Factor XIII to mammalian tissue.

16. The method of claim 15, wherein the mammalian tissue is human tissue.

17. The method according to claim 1, wherein the prothrombin activator comprises an extract of fish gills or a combination of fish egg and fish gills.

18. The method according to claim 1, wherein the extract comprises the prothrombin activator in a ratio between fish egg and fish gills in the range of 0:9 to 9:1.

19. The method according to claim 1, wherein the extract comprises the prothrombin activator in a ratio between fish egg and fish gills of 1:3.

20. The method according to claim 1, wherein the activation process in step (iv) is carried out at 0-40° C.

21. The method according to claim 1, wherein the activation process in step (iv) is carried out under gentle stirring for 30 second to 48 hours.

22. The method according to claim 1, wherein the activation process in step (iv) is terminated after a period in the range of 10 minutes to 10 hours.

23. The method according to claim 1, wherein the activation process in step (iv) is terminated by addition of sodium oxalate in a concentration of 0.01 to 0.5.

24. The method according to claim 1, wherein the activated thrombin obtained in step (iv) is centrifuged.

25. The method according to claim 23, wherein the supernatant obtained is filtered.

26. A thrombin preparation obtainable by a method according to claim 1.

27. A prothrombin activator for activating prothrombin to thrombin comprising extract of fish egg, fish gills or a combination thereof.

28. The prothrombin activator according to claim 27, wherein the ratio between fish egg and fish gills in the extract is in the range of 0:9 to 9:1.

29. The prothrombin activator according to claim 27, wherein the ratio between fish egg and fish gills in the extract is 1:3.

30. A method for producing a fibrin sealant, said method comprising the steps of:
(i) providing a fibrinogen and Factor XIII,
(ii) providing a thrombin, where said thrombin has been activated using the prothrombin activator of claim 27, and
(iii) combining the fibrinogen and Factor XIII of step (i) and the thrombin of step (ii) to form the fibrin sealant.

31. The method according to claim 30, wherein at least one of the fibrinogen, Factor XIII or the thrombin is extracted from plasma.

32. The method according to claim 31, wherein the plasma is separated from whole blood which is drawn from a donor fish or a mammal.

33. The method according to claim 30, wherein a salt is added to the combined thrombin and fibrinogen and Factor XIII to form the fibrin sealant.

34. The method according to claim 33, wherein the salt is a calcium salt.

35. The method according to claim 32, wherein the donor fish is a cold water fish.

36. The method according to claim 35, wherein the donor fish is selected from the group consisting of a salt-water fish, a brackish-water fish and a fresh-water fish.

37. A sealant obtainable by a method according to claim 32.

38. The method according to claim 12, wherein the salt water fish belongs to the genus Salmonidae.

39. The method according to claim 12, wherein the salt water fish is Salmo salar.

40. The method according to claim 1, wherein the activation process in step (iv) is carried out at 4-25° C.

41. The method according to claim 1, wherein the activation process in step (iv) is terminated by addition of sodium oxalate in a concentration of 0.04-0.1.

42. The method according to claim 36, wherein the salt water fish belongs to the genus Salmonidae.

43. The method according to claim 36, wherein the salt water fish is an Atlantic salmon (Salmo salar), a rainbow trout or a white fish.

44. The method according to claim 36, wherein the salt water fish is cod or halibut.
